(19) United States
(12) Patent Application Publication
(10) Pub. No.: US 2022/0296156 A1
(43) Pub. Date: Sep. 22, 2022

(54) NEUROMONITORING DEVICE

(71) Applicant: JIANGSU BAINING YINGCHUANG MEDICAL TECHNOLOGY CO., LTD., Naning (CN)

(72) Inventors: Zhigang SHI, Nanjing (CN); Jiancong LI, Nanjing (CN)

(73) Assignee: JIANGSU BAINING YINGCHUANG MEDICAL TECHNOLOGY CO., LTD., Nanjing (CH)

(21) Appl. No.: 17/805,858

(22) Filed: Jun. 7, 2022

Related U.S. Application Data
(63) Continuation-in-part of application No. 16/699,847, filed on Dec. 2, 2019.
Application No. 16/699,847 is a continuation of application No. PCT/CN2019/086104, filed on May 9, 2019.

(30) Foreign Application Priority Data
Aug. 1, 2018 (CN) 201810863181.9

(51) Int. Cl. A61B 5/388; A61B 5/00; A61B 5/389; A61B 34/20

(52) U.S. Cl. CPC A61B 5/388; A61B 5/742; A61B 5/389; A61B 34/20

(57) ABSTRACT

The present disclosure relates to a neuromonitoring device. The neuromonitoring device may include a probe, an operation part, and a display part. The probe may include a probe head, an elastic piece, and an elastic measuring piece. The probe head may be connected to the elastic piece. The elastic measuring piece may be connected to the elastic piece and may be used to measure an elasticity value of the elastic piece and convert the elasticity value into a first electrical signal. The display part may be configured to display prompt information. The prompt information may include prompt information regarding the elasticity value determined based on the first electrical signal.

### CROSS-REFERENCE TO RELATED APPLICATIONS

This application is a continuation-in-part of U.S. Ser. No. 16/699,847, filed on Dec. 2, 2019, which is a continuation of International Application No. PCT/CN2019/086104, filed on May 9, 2019, which claims priority of Chinese Patent Application No. 201810863181.9, entitled “A NEUROMONITORING PROBE FOR SENSING CONTACT STATE,” filed on Aug. 1, 2018, the entire contents of which are incorporated herein by reference.

### TECHNICAL FIELD

The present disclosure relates to the field of medical devices, and in particular, to a neuromonitoring device.

### BACKGROUND

Neuromonitoring probes are often used during surgery. The neuromonitoring probe is usually connected to a neuro monitor and a surgeon uses the neuromonitoring probe to locate and identify nerves at risk in a surgical area, thus nerves can be protected from injury during the operations. For existing neuromonitoring probes, there may be some problems such as inconvenience in operation, difficulty in controlling the strength and the magnitude of the stimulation current, etc. Therefore, it is desirable to provide an improved neuromonitoring device.

### SUMMARY

The present disclosure provides a neuromonitoring device. The device may include a probe, an operation part, and a display part. The probe may include a probe head, an elastic piece, and an elastic measuring piece. The probe head may be connected to the elastic piece. The elastic measuring piece may be connected to the elastic piece and may be used to measure an elasticity value of the elastic piece and convert the elasticity value into a first electrical signal. The display part may be configured to display prompt information. The prompt information may include prompt information regarding the elasticity value determined based on the first electrical signal.

In some embodiments, the operation part may include a handle. The probe may be fixedly connected to the handle, and the display part may be set on the handle.

In some embodiments, the prompt information may include a magnitude of a nerve stimulation current.

In some embodiments, the neuromonitoring device may further include a probe monitoring part configured to monitor a usage status of the probe and generate probe monitoring information. The usage status of the probe includes a cumulative usage time of the probe and/or an elastic condition of the elastic piece.

In some embodiments, the probe may further include a sleeve. The elastic piece may be installed in the sleeve. An end of the probe head may be inserted into a first end of the sleeve to connect to the elastic piece and a second end of the sleeve may be connected to the handle.

In some embodiments, the neuromonitoring device may further include a fixing device and a driving device. The operation part may be configured to receive a control operation. The driving device may be configured to drive the probe to move based on the control operation, and the fixing device may be configured to fix positional relationship between the driving device and an object.

In some embodiments, the neuromonitoring device may further include a controller configured to adjust action instructions sent to the driving device when a movement range or speed of the control operation does not meet a preset condition.

In some embodiments, the neuromonitoring device may further include an electromyography measuring piece configured to measure a second electrical signal. The second electrical signal may be an electromyographic signal of the object of the probe.

In some embodiments, the prompt information may include electromyographic information based on the second electrical signal.

In some embodiments, the present disclosure provides a neuromonitoring method implemented by a nerve detection device. The method may include: receiving a control operation from an operation part; and determining action instructions and sending the action instructions to a driving device to drive a probe to move.

In some embodiments, the method may further include adjusting the action instructions sent to the driving device when a movement range or speed of the control operation does not meet a preset condition.

In some embodiments, the method may further include measuring a second electrical signal, wherein the second electrical signal is an electromyographic signal of an object of the probe.

In some embodiments, the method may further include displaying prompt information, wherein the prompt information includes electromyographic information based on the second electrical signal.

In some embodiments, the method may further include determining a first sensitivity coefficient of the object; determining a second sensitivity coefficient of a nerve; and determining a maximum elasticity value and a maximum current value of a current adjustment part based on the first sensitivity coefficient and the second sensitivity coefficient.

In some embodiments, the determining a first sensitivity coefficient of the object may include: obtaining relevant information of the object, the relevant information including occupational information and/or a sound pressure level of speech; and determining, based on the relevant information of the object, the first sensitivity coefficient of the object.

In some embodiments, the method may further include: obtaining a first detection parameter, at least one group of second detection parameters and a feedback signal corresponding to the at least one group of second detection parameters; and processing the first detection parameter, the at least one group of second detection parameters and a feedback signal corresponding to the at least one group of the second detection parameters based on a prediction model to output prediction feedback corresponding to the first detection parameter, wherein the prediction model is a machine learning model.

In some embodiments, the method may further include: processing the first detection parameter, the at least one group of second detection parameter and a feedback signal corresponding to the at least one group of the second detection parameters based on the prediction model to output a prediction reference point, wherein the prediction reference point is a point in the object related to the feedback signal; and the prediction reference point is configured to display.

In some embodiments, for one of the at least one group of second detection parameters, the second detection parameter includes: an operating pressure, an operating current, and an operating position. The method may further include determining, based on a distance between the operating position and a predicted historical reference point corresponding to the second detection parameter, a position reference degree of the second detection parameter; and processing the first detection parameter, the at least one group of second detection parameters and a feedback signal corresponding to the at least one group of second detection parameters, the at least one group of the second detection parameters and the corresponding position reference degree based on the prediction model to output the prediction reference point.

In some embodiments, the method may further include: determining warning information according to the prediction feedback; and displaying the warning information.

The present disclosure may further provide a computer-readable storage medium storing computer instructions. When reading the computer instructions in the storage medium, a computer may implement the method including: receiving a control operation from an operation part; and determining action instructions and sending the action instructions to a driving device to drive a probe to move.

### BRIEF DESCRIPTION OF THE DRAWINGS

The present disclosure is further described in terms of exemplary embodiments. These exemplary embodiments are described in detail with reference to the drawings. These embodiments are not limiting, and in these embodiments, like reference numerals represent similar structures, and wherein:

FIG. 1 is a schematic diagram illustrating a profile of a neuromonitoring device according to some embodiments of the present disclosure;

FIG. 2 is a schematic diagram illustrating a structure of a neuromonitoring device according to some embodiments of the present disclosure; and

FIG. 3 is a schematic diagram illustrating a connection structure of a probe head and a sleeve according to some embodiments of the present disclosure.

FIG. 4 is a schematic diagram illustrating a structure of a driving device and a fixing device according to some embodiments of the present disclosure;

FIG. 5 is a flowchart illustrating an exemplary process for monitoring a nerve according to some embodiments of the present disclosure;

FIG. 6 is a structural diagram illustrating an exemplary prediction model according to some embodiments of the present disclosure; and

FIG. 7 is a structural diagram illustrating an exemplary neuromonitoring device according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless apparent from the locale or otherwise stated, like reference numerals represent similar structures or operations throughout the several views of the drawings.

As used in the disclosure and the appended claims, the singular forms “a,” “an,” and “the” include plural forms as well unless the content clearly indicates otherwise. In general, the terms “comprise,” “comprising,” “include,” and/or “including” when used in this disclosure, specify the presence of stated steps and elements, and these steps and elements do not constitute an exclusive listing. The methods or devices may also include other steps or elements.

Although the present disclosure makes various references to certain modules in the system according to some embodiments of the present disclosure, any number of different modules may be used and run on a client terminal and/or a server. The modules are merely illustrative, and different aspects of the system and method may be implemented by different modules.

The flowcharts used in the present disclosure illustrate operations that systems implement according to some embodiments of the present disclosure. It should be understood that the preceding or following operations may be implemented not in order. Conversely, the operations may be implemented in an inverted order, or simultaneously. Moreover, one or more other operations may be added to the flowcharts. One or more operations may be removed from the flowcharts.

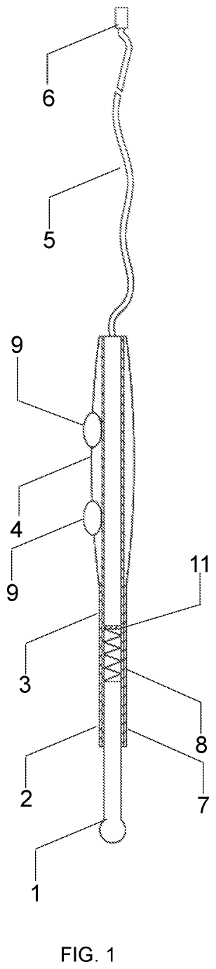

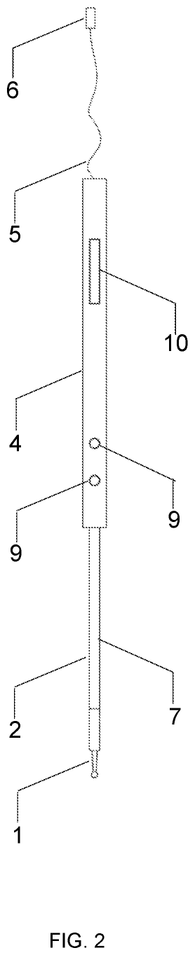

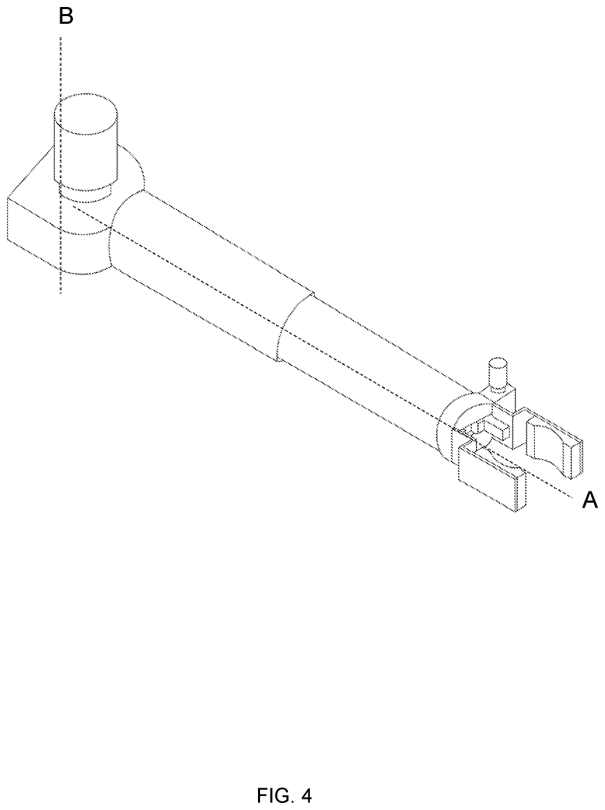

In some embodiments, a neuromonitoring device may include a probe, an operation part, and a display part. The probe is a part used to probe the nerve. The probe may include a probe head, an elastic piece and an elastic measuring piece, and may also include other components. The probe head may be connected to the elastic piece; the elastic measuring piece may be connected to the elastic piece for measuring the elasticity value of the elastic piece and converting the elasticity value into a first electrical signal. Regarding the structure of the probe, reference may be made to the description of FIG. 1 and FIG. 2, but is not limited by FIG. 1 and FIG. 2. The probe can be connected to the handle as shown in FIGS. 1 and 2, or be connected to the driving device as shown in FIG. 4. The display part may be used for displaying prompt information. The prompt information may include prompt information about the elasticity value determined according to the first electrical signal. The display part may include the elasticity prompt part shown in FIG. 1 and FIG. 2, or may be an independent display screen or other components that can display information.

FIG. 1 is a schematic diagram illustrating a profile of a neuromonitoring device according to some embodiments of the present disclosure. FIG. 2 is a schematic diagram illustrating a structure of a neuromonitoring device according to some embodiments of the present disclosure. The neuromonitoring device may include a handle 4, a probe 7, and an elastic prompt 10. The probe 7 may be connected to the handle 4. The probe 7 may include a probe head 1, an elastic piece 8, and an elastic measuring piece 11. The probe head 1 may be connected to the elastic piece 8. When the neuromonitoring device is being used, the probe head 1 may contact with a human body (e.g., nerves, tissues) and may receive a pressure given by the human body. Further, the probe head 1 may transmit the pressure to the elastic piece 8 and then the elastic piece 8 may be elastically deformed, causing the probe head 1 to move. The probe head 1 is retractable due to the elastic deformation of the elastic piece 8, so that it can contact with the human body continuously and reliably. In addition, when using the neuromonitoring device of the present disclosure, a user may sense a resilience force and then sense a pressure exerted by the probe 7 on the human body, so that the user can control an operation strength of using the neuromonitoring device to ensure a reliable contact between the probe 7 and the nerves or the tissues. The elastic measuring piece 11 may be connected to the elastic piece 8 and may be used to measure a first elasticity value of the elastic piece 8 and convert the elasticity value into an electrical signal. The elastic prompt 10 may be connected to the elastic measuring piece 11 and may be used to receive the first electrical signal regarding the elasticity value of the elastic piece 8 generated by the elastic measuring piece 11 and generate prompt information regarding the elasticity value of the elastic piece 8 based on the first electrical signal. The elastic prompt 10 may prompt the elasticity value of the elastic piece 8 in various forms, including but not limited to texts, images, voices, etc.

As illustrated in FIG. 2, the elastic prompt 10 may be set on the handle 4. In some embodiments, the elastic prompt 10 may include a display screen used to display the elasticity value. In some embodiments, when the elasticity value exceeds a set threshold, the elastic prompt 10 may provide an alert (e.g., displaying a warning image, providing a warning tone) to remind the user to control an operation strength. The set threshold may be a fixed value or may be determined based on different kinds of nerves to be detected. Merely for example, for cranial nerves, since the cranial nerves are relatively sensitive, the threshold may be set as a relatively low value (e.g., 0.8N); for laryngeal nerves, the threshold may be set as 1.2N; for nerves at a face, a hand, a foot, or a knee, the threshold may be set as 3N.

In some embodiments, the neuromonitoring device of the present disclosure may further include a monitor (not shown). In some embodiments, one end of a wire 5 may be connected to the probe 7 and the other end may be connected to the monitor through a socket 6. In some embodiments, the elastic prompt 10 may be set in the monitor. Specifically, the monitor may receive the electrical signal regarding the elasticity value of the elastic piece 8 generated by the elastic measuring piece 11 and generate prompt information regarding the elasticity value of the elastic piece 8. For example, the monitor may include a display screen through which the elasticity value may be displayed. In addition to a text display, the monitor may also prompt the elasticity value by means of images, voices, etc. Since the elastic prompt 10 is used, when using the neuromonitoring device of present disclosure, the user (e.g., a doctor) can conveniently know a pressure applied to a patient and then control the operation strength, which can ensure a reliable contact between the probe 7 and the nerves or the tissues, and also can protect the nerves or the tissues of the patient from injury.

In some embodiments, different types of neuromonitoring devices may have different maximum elasticity values. For example, elastic pieces with different elastic coefficients may be used to achieve the differentiation of the maximum elasticity value. Specifically, according to Hooke's law:

F=k×X (1)

Where F refers to an elasticity value of an elastic piece, k refers to an elastic coefficient of the elastic piece, and X refers to an elastic deformation of the elastic piece. According to equation (1), for elastic pieces with different elastic coefficients k, when a same elastic deformation X occurs, elasticity values F are different. Accordingly, in a situation that the maximum elastic deformation is fixed, different maximum elasticity values can be achieved by selecting elastic pieces with different elastic coefficients. In some embodiments, neuromonitoring devices with different maximum elasticity values may be used for different types of surgery. For example, for nerves with a relatively high sensitivity, a neuromonitoring device with a relatively small maximum elasticity value may be used; for nerves with a relatively low sensitivity, a neuromonitoring device with a relatively high maximum elasticity value may be used. Merely for example, for cranial nerves, a neuromonitoring device with a maximum elasticity value of 0.8N may be used; for laryngeal nerves, a neuromonitoring device with a maximum elasticity value of 1.2N may be used; for nerves at a face, a hand, a foot, or a knee, a neuromonitoring device with a maximum elasticity value of 3N may be used. In some embodiments, neuromonitoring devices with different maximum elasticity values may be used for different individuals. For example, for patients with a relatively high sensitivity, a neuromonitoring device with a relatively small maximum elasticity value may be used; for patients with a relatively low sensitivity, a neuromonitoring device with a relatively high maximum elasticity value may be used.

The elastic measuring piece 11 may convert the elasticity value of the elastic piece 8 into an electrical signal. In some embodiments, the elastic measuring piece 11 may include an adjustable resistor connected to the elastic piece 8, whose resistance may change with a change of a length of the elastic piece 8, thereby realizing the conversion of the elasticity value to the electrical signal. For example, the elasticity value may be positively related to the resistance value; or the elasticity value may be inversely related to the resistance value. In some embodiments, the elastic measuring piece 11 may include a pressure sensor which may measure the elasticity value of the elastic piece 8. Specifically, when the neuromonitoring device is being used, when the probe head 1 is in contact with the human body and receives the pressure given by the human body, the elastic piece 8 may be compressively deformed and exert a pressure on the pressure sensor, then the elasticity value of elastic piece 8 may be obtained based on a pressure value measured by the pressure sensor.

In some embodiments, the elastic piece 8 may be also connected to an elastic adjustment part (not shown) used to adjust the maximum elasticity value of the elastic piece 8. For example, the maximum elasticity value may be adjusted and an elastic force may be changed by limiting the stretchable length of the elastic piece 8. For different types of surgery, the maximum elasticity value of the elastic piece 8 may be adjusted by the elastic adjustment part to match a maximum elasticity value corresponding to a type of surgery. For example, for cranial nerves, the maximum elasticity value of elastic piece 8 may be adjusted to 0.8N; for laryngeal nerves, the maximum elasticity value may be adjusted to 1.2N; for nerves at a face, a hand, a foot, or a knee, the maximum elasticity value may be adjusted to 3N.

In some embodiments, the elastic piece 8 may be made of a conductive material. The conductive material may include a metal, a conductive rubber, a conductive non-metal, a conductive alloy, or the like, or a combination thereof. In some embodiments, the maximum elasticity value of the elastic piece 8 may be also adjusted for different individuals. For example, for patients with a relatively high sensitivity, the maximum elasticity value may be decreased; for patients with a relatively low sensitivity, the maximum elasticity value may be increased.

In some embodiments, the handle 4 may be also provided with a current adjustment part 9 used to regulate a magnitude of a nerve stimulation current. In some embodiments, the current adjustment part 9 may be electrically connected to the monitor through a wire. After receiving a current adjustment signal sent by the current adjustment part 9, the monitor may control the magnitude of the output stimulation current. For example, the monitor may include a host and a current output unit. The host may be used to receive the current adjustment signal sent by the current adjustment part 9, generate a current control signal based on the current adjustment signal, and send the current control signal to the current output unit. The current output unit may output a current of a corresponding magnitude based on the received current control signal. In some embodiments, the current output unit may include a voltage/current conversion integrated circuit which can convert an input voltage into an output current. Specifically, after the host of the monitor receives the current adjustment signal, a microcontroller unit (MCU) of the host may control the input voltage of the voltage/current conversion integrated circuit by controlling a pulse width modulation (PWM) wave. The voltage/current conversion of the integrated circuit may output a current with an appropriate magnitude.

In some embodiments, for different types of nerves, stimulation currents of different magnitudes may be obtained by adjustment. For example, for cranial nerves, the stimulation current may be adjusted to 0-0.5 mA; for laryngeal nerves, the stimulation current may be adjusted to 0.5 mA-10 mA; for nerves at a face, a hand, a foot, or a knee, the stimulation current may be adjusted to 10 mA-30 mA. In some embodiments, due to differences in sensitivity of different individuals, the stimulation currents of different magnitudes may be obtained by adjustment for different individuals. For example, for patients with a relatively high sensitivity, the stimulation current may be decreased; for patients with a relatively low sensitivity, the stimulation current may be increased.

In some embodiments, a maximum current threshold may be set to limit the stimulation current from exceeding the maximum current threshold, thereby ensuring the safety of detecting nerves or tissues. For example, the maximum current threshold may be 40 mA, 35 mA, 30 mA, 25 mA, 20 mA, etc. In some embodiments, different maximum current thresholds may be set for different types of nerves. For example, for cranial nerves, the maximum current threshold may be set as 0.5 mA; for laryngeal nerves, the maximum current threshold may be set as 10 mA; for nerves at a face, a hand, a foot, or a knee, the maximum current threshold may be set as 30 mA. In some embodiments, different maximum current thresholds may be set for different individuals. For example, for patients with a relatively high sensitivity, the maximum current threshold may be set as a relatively low value; for patients with a relatively low sensitivity, the maximum current threshold may be set as a relatively high value.

The current adjustment part 9 may be in various forms including but not limited to a button, a knob, a touch key, etc. In some embodiments, as illustrated in FIG. 1 and FIG. 2, the current adjustment part 9 may be two buttons used to increase and decrease the current respectively. An adjustment step size may be a fixed value or a changing value. In some embodiments, different adjustment step sizes may be set for different stimulation current ranges. It can be understood that for a relatively small stimulation current, an adjustment precision requirement is relatively high so that a relatively small adjustment step size may be set to achieve a high-precision adjustment; for a relatively large stimulation current, the adjustment precision requirement is relatively low so that a relatively large step size may be set to achieve a rapid adjustment. For example, for a range from 0 to 0.5 mA, the adjustment step size may be 0.01 mA; for a range from 0.5 mA to 1 mA, the adjustment step size may be 0.1 mA; in the range of 1 mA to 10 mA, the adjustment step size may be 0.5 mA; for a range from 10 mA to 30 mA, the adjustment step size may be 1 mA. It should be noted that the two buttons illustrated in FIG. 1 and FIG. 2 are an example of the current adjustment part, and are not intended to limit the present disclosure. In some embodiments, current adjustment parts of other forms may be set. For example, four buttons may be set, two of which are used to roughly adjust (increase or decrease) the stimulation current based on a first step size, and the other two are used to finely adjust the stimulation current based on a second step size, wherein the second step size is less than the first step size.

In some embodiments, the neuromonitoring device of the present disclosure may also include a stimulation current prompt used to prompt the magnitude of the stimulation current. The magnitude of the stimulation current may be prompted in various forms including but not limited to texts, images, voices, etc. In some embodiments, the stimulation current prompt may be set on the handle 4. For example, a display screen may be set on the handle 4 and may be used to display the magnitude of stimulation current. In some embodiments, the stimulation current prompt and the elasticity value prompt described above may be integrated as a same component; or both may be separate components. In some embodiments, the stimulation current prompt may be set on the monitor. For example, the display screen of the monitor may display the magnitude of stimulation current.

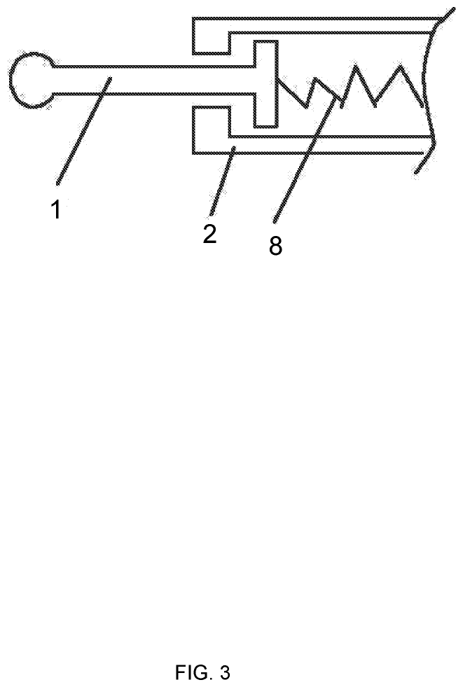

In some embodiments, the probe 7 may also include a sleeve 2. FIG. 3 is a schematic diagram illustrating a connection structure of a probe head 1 and a sleeve 2 according to some embodiments of the present disclosure. As illustrated in FIG. 1 and FIG. 3, the elastic piece 8 may be installed in the sleeve 2. One end of the probe head 7 may be inserted into a first end of the sleeve 2 to connect to the elastic piece 8 and a second end of the sleeve 2 may be connected to the handle 4. In some embodiments, the sleeve 2 may be made of a conductive material and the wire 5 may be electrically connected to the sleeve 2, thereby achieving an electrical connection between the wire 5 and the probe 7. In some embodiments, a surface of the sleeve 2 may be provided with an insulation layer 3 which may be a structure such as a heat shrinking sleeve, an insulating coating, etc. In some embodiments, the probe head 1 may be a ball-head structure. In some embodiments, in order to prevent the probe head 1 from slipping out of the sleeve 2, in addition to a manner that the probe head 1 is welded to the elastic piece 8, a non-slip step may be provided at one end of the probe head 1 inserted into the sleeve 2 and a matching limit step is provided on an inner wall of the sleeve 2. At the time of installation, the probe head 1 may be inserted into the sleeve 2 from the other end of the sleeve 2, and after the end of the probe head 1 provided with the step is in contact with the step inside the sleeve 2, the head of the probe head 1 may be spherically roughened. In addition, after the end of the probe head 1 provided with the step is inserted into the sleeve 2, an end portion of the sleeve 2 may be turned inward to form an inside step.

In some embodiments, the neuromonitoring device of the present disclosure may also include a probe monitoring part (not shown) used to monitor a usage status of the probe 7 and generate probe monitoring information. For example, the probe monitoring part may monitor a cumulative usage time of the probe. Merely for example, the probe monitoring part may read/write the cumulative usage time of the probe by an electrically erasable programmable read only memory (EEPROM). As another example, the probe monitoring part may monitor an elastic condition of the elastic piece in the probe. In some embodiments, in response to that the probe monitoring information satisfies a set condition, the probe monitoring part may provide a prompt. For example, when the cumulative usage time exceeds a certain time period or the elastic condition of the elastic piece decays to a certain extent, the probe monitoring part may provide an alarm to prompt the user to replace the elastic piece in time. In some embodiments, the probe monitoring part may be set on the handle 4. In some embodiments, the probe monitoring part may be integrated into the monitor.

In some embodiments, the neuromonitoring device of the present disclosure may also include an operation part, a fixing device and a driving device.

The operation part may be used to receive a control operation, and the driving device may be used to drive the probe to move based on the control operation. In some embodiments, the driving device may be physically and/or signally connected to the operation part. For example, the operation part may include a remote control handle. The operation part may be electrically connected with the driving device so that the driving device can drive the neuromonitoring device to move based on the control operation received by the operation part. Effects of remote control of the neuromonitoring device and automatic prevention of vibration of the neuromonitoring device can be achieved through the driving device. For example, medical staff may control a movement direction, a movement speed, or a movement range, etc. of the probe by operating the remote control handle. The fixing device is used to fix the positional relationship between the driving device and the object. For example, the fixing device may fix the drive device to the head of a measured person and keep the mouth open. As a result, the positioning accuracy can be maintained.

FIG. 4 is a schematic diagram illustrating a structure of a driving device and a fixing device according to some embodiments of the present disclosure.

In some embodiments, the neuromonitoring device may further include a controller. The controller may be a component with functions of data processing, calculation, etc., such as a CPU and other related components. In some embodiments, the controller may be used to adjust the action instructions sent to the driving device when a movement range or speed of the control operation does not meet a preset condition. The preset condition may be a manually set speed threshold. For example, when the movement range or movement speed of the neuromonitoring device exceeds the preset threshold, the controller may send an action instruction to reduce the movement range or reduce the movement speed to the driving device to ensure the stability of the neuromonitoring device.

In some embodiments, the controller may be electrically connected with the driving device, a current adjustment part, and a elasticity adjustment part to transform a first detection parameter into a second electrical signal, and send the second electrical signal to the driving device, the current adjustment part, and the elasticity adjustment part.

In some embodiments, the controller may be electrically connected with the elastic measuring piece to receive a first electrical signal sent by the elastic measuring piece.

The first detection parameter may refer to a plurality of groups of detection parameters stored in the operation part that have not been actually executed during the detection process. Each group of first detection parameters may correspond to a plurality of detection parameters that detection is performed at one position. In some embodiments, the first detection parameters may include a position of a neuromonitoring probe, a set pressure value, a magnitude of a stimulation current, or the like. The position of the neuromonitoring probe may be obtained through the driving device. In some embodiments, the first detection parameters may be determined by medical staff.

The second electrical signal may refer to an electrical signal obtained from the first detection parameters after signal conversion.

In some embodiments, the neuromonitoring device may also include an electromyography measuring piece used to measure the second electrical signal. The second electrical signal may be an electromyographic signal of an object of the probe. It may be understood that the electromyography measuring piece may be used as a part of the probe in the neuromonitoring device, or may be another part independent of the probe.

In some embodiments, the prompt information may include electromyographic information based on the second electrical signal.

The electromyography measuring piece may be used to collect biological electromyographic signals. Electromyographic signals is a superposition of action potentials of motor units in many muscle fibers in time and space, which may reflect neuromuscular activity to a certain extent.

In some embodiments, the electromyography measuring piece may include a signal acquisition device used to acquire electromyographic signals. In a specific embodiment, the signal acquisition device may include an electromyographic electrode patch. During use, the electromyographic electrode patch may be attached to a position of target muscle group to collect electromyographic signals generated by the target muscle group. In a specific embodiment, the signal acquisition device may include an electromyographic electrode needle. During use, the electromyographic electrode needle may be punctured into a living body, so that the needle head may contact a deep nerve or muscle tissue, thereby completing the acquisition of electromyographic signals.

In some embodiments, the controller may be electrically connected with the electromyography measuring piece to receive electromyographic signals sent by the electromyography measuring piece.

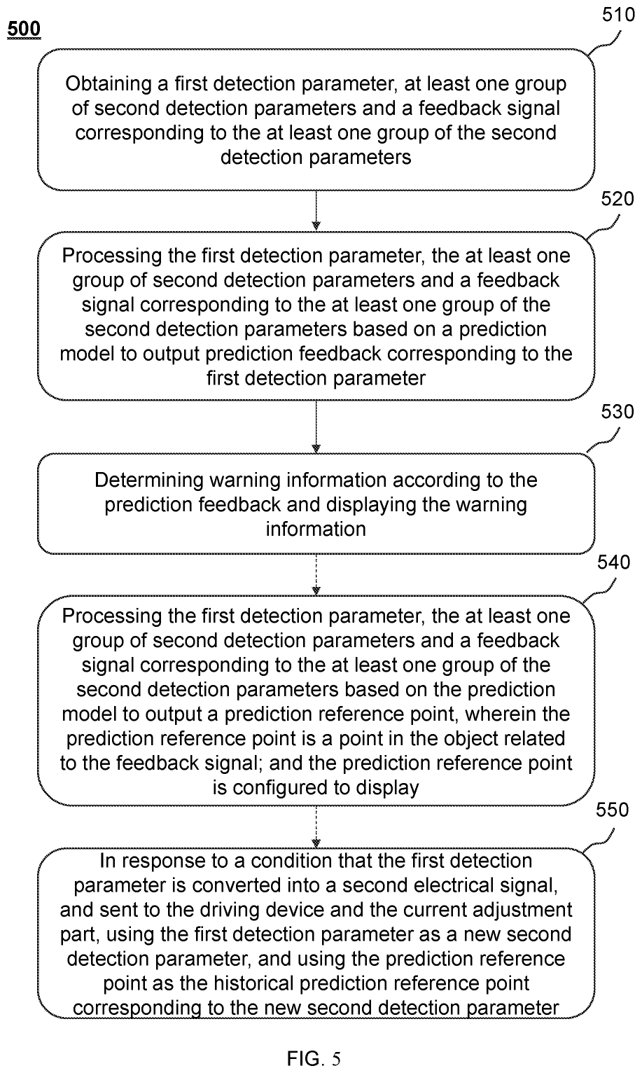

FIG. 5 is a flowchart illustrating an exemplary process for monitoring a nerve according to some embodiments of the present disclosure. In some embodiments, the process 500 may be performed by a controller 740 in a neuromonitoring device 700. As shown in FIG. 5, the process 500 may include the following operations.

In 510, a first detection parameter, and at least one group of second detection parameters and a corresponding feedback signal (that is, a feedback signal corresponding to the at least one group of the second detection parameters) may be obtained.

The second detection parameter may refer to a plurality of groups of detection parameters obtained by the neuromonitoring device during a detection process. The detection parameters may include a position of a neuromonitoring probe (which may be determined by a controller), a pressure reading (which may be obtained by an elastic measuring piece), a magnitude of a stimulation current (which may be determined by the controller, and/or a feedback signal (which may be determined by electromyography measuring piece). Each group of second detection parameters may correspond to a plurality of detection parameters obtained at one position. For example, the second detection parameter may include an operating pressure, an operating current, and an operating position. The operating pressure may be a probed pressure reading corresponding to the second detection parameter. The operating current may be a magnitude of a probed stimulation current corresponding to the second detection parameter. The operating position may be a position of a probed neuromonitoring probe corresponding to the second detection parameter.

The feedback signal may refer to feedback information generated by a part of an object obtained by the neuromonitoring device when the detection is performed on the part of the object. The feedback signal may include electromyographic signals and pressure signals generated after human tissue or nerves are stimulated by a current. One detection may correspond to a group of feedback signals. For example, the probe may be placed at a certain part of a human body, and a certain pressure and a stimulation current may be applied to the part. In this case, electromyographic signals obtained by the electromyography measuring piece and the second electrical signal obtained by the elastic measuring piece may be used as the feedback signals corresponding to the detection.

In some embodiments, whether the nerve is detected may be determined based on whether the strength of the electromyographic signal emitted by the nerve reached a detection threshold. For example, when the strength of the electromyographic signal emitted by the nerve is greater than the detection threshold, it can be considered that the nerve is detected. The detection threshold may be predetermined based on a type of nerve, patient sensitivity, or the like. For example, when sensitivity of an object is relatively high and a nerve is a cranial nerve, a detection threshold of the electromyographic signal may be set to a lower value. For example, the detection threshold may be set to 50 mv. When sensitivity of an object is relatively low and nerves are nerves in a face, hands, feet, and knees, a detection threshold of the electromyographic signal may be set to a higher value. For example, the detection threshold may be set to 100 mv.

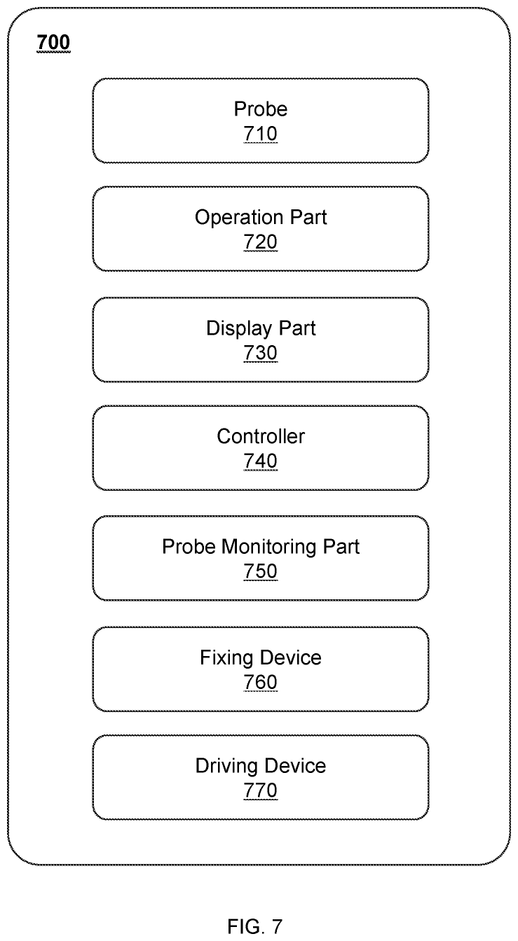

In some embodiments, the operation part 720 may store the first detection parameter. The controller 740 may store a plurality of groups of second detection parameters and corresponding feedback signals. The controller 740 may obtain at least one group of second detection parameters and a corresponding feedback signal, and obtain the first detection parameter from the operation part 720. For detailed descriptions of the operation part 720 and the controller 740, please refer to FIG. 7 and relevant descriptions thereof.

In 520, the first detection parameter, the at least one group of second detection parameters and the corresponding feedback signal may be processed based on the prediction model to output prediction feedback corresponding to the first detection parameter. The prediction model may be a machine learning model.

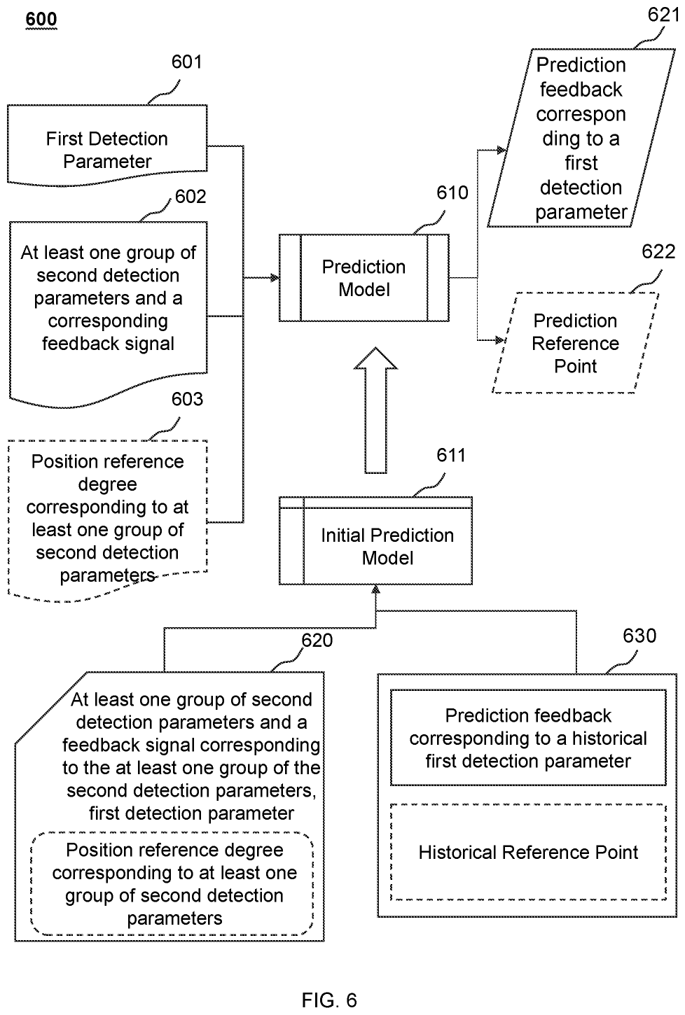

The prediction model may be used to determine the feedback signal corresponding to the first detection parameter. In some embodiments, the prediction model may be a machine learning model. For example, the prediction model may be a Transformer model. The Transformer model is a machine learning model based on a self-attention mechanism. The self-attention mechanism may take a relationship within source data or within output data during a model prediction. The Transformer model may include an encoder layer and a decoder layer. The structure of the Transformer model may enable the model to be trained in parallel and have global information. In some embodiments, the Transformer model used may be a BERT model, or the like. For detailed descriptions of the prediction model, please refer to FIG. 6 and relevant descriptions thereof.

In 530, warning information according to the prediction feedback may be determined, and the warning information may be displayed.

The warning information may refer to information used to prompt a user that there is a risk in detection corresponding to the first detection parameter. The warning information may be issued to the user in various ways, including but not limited to displaying through a display part, voice broadcasting, handle vibration, etc. For detailed descriptions of the display part 730, please refer to FIG. 7 and relevant descriptions thereof.

In some embodiments, the controller 740 may determine the warning information based on a magnitude of the prediction feedback corresponding to the first detection parameter output by the prediction model. For example, the processor 730 may set a safety range for prediction feedback. When the prediction feedback corresponding to the first detection parameter exceeds the safety range, the controller 740 may generate warning information. The warning information may be to prompt a user that the prediction feedback corresponding to the first detection parameter has exceeded the safety range. Further, the warning information may also include a percentage of the prediction feedback corresponding to the first detection parameter that exceeds the safety range, and an adjustment amount of detection parameter suggested to the user.

In 540, the first detection parameter, the at least one group of second detection parameters and the corresponding feedback signal may be processed, based on the prediction model, to output the prediction reference point. The prediction reference point is a point of the object related to the feedback signal, and the prediction reference point is used for display.

The prediction reference point may refer to a point in the object related to the feedback signal. The prediction reference point may refer to a reference point obtained based on the model prediction. In some embodiments, the prediction reference point may be a point where a nerve can be detected and is close to the position of a neuromonitoring probe corresponding to the first detection parameter. That a nerve can be detected refers to that when the neuromonitoring probe is placed into the prediction reference point, an intensity of the electromyographic signal emitted by the nerve is greater than or equal to a detection threshold. The prediction reference point may be obtained based on a trained prediction model. For more descriptions of the prediction model, please refer to FIG. 7 and relevant descriptions thereof.

The prediction reference point may be displayed. For example, the display part 730 may be used to display a prediction reference point to a user. In some embodiments, the display part 730 may also be used to display different predicted historical reference points in different colors. For example, the display part 730 may display different predicted historical reference points in different shades of color, and the predicted historical reference point whose prediction time is closer to a current time may have a darker color.

In some embodiments, the prediction model may process the first detection parameter, the at least one group of second detection parameters and the corresponding feedback signal, the at least one group of second detection parameters and the corresponding position reference degree based on the prediction model to output the prediction reference point. For one of the at least one group of second detection parameters, the second detection parameter may include an operating pressure, an operating current, and an operating position. In some embodiments, the controller 740 may determine, based on a distance between the operating position and a predicted historical reference point corresponding to the second detection parameter, a position reference degree of the second detection parameter.

The position reference degree may be a degree to which the second sounding parameter may be referred to in determining the prediction reference point and the prediction feedback. The greater the reference degree of the second detection parameter, the greater the position reference degree of the second detection parameter.

In some embodiments, the position reference degree corresponding to each second detection parameter may be periodically updated based on a same preset trigger condition. In some embodiments, the preset trigger condition may be: a new prediction reference point can be obtained through the prediction model.

In some embodiments, the position reference degree of the second detection parameter may be determined based on the distance between the operating position and the predicted historical reference point corresponding to the second detection parameter. The predicted historical reference point may refer to a prediction reference point has been output by the prediction model. In some embodiments, a distance between the operating position in the second detection parameter and the at least one predicted historical reference point may be calculated respectively to obtain at least one distance. A position reference degree of the second detection parameter may be determined based on a shortest distance among the at least one distance.

For example, a position reference degree may be determined based on equation (2).

$$r = \frac{k}{d} \qquad ( 2 )$$

where r denotes a position reference degree; d denotes a minimum value of a distance between an operating position in the second detection parameter and at least one predicted historical reference point; K denotes a preset parameter for adjusting the position reference degree. K may be determined based on experience. For example, K may be 1. It can be seen from the equation (2) that the smaller the minimum value of the distance between the operating position in the second detection parameter and the at least one predicted historical reference point, the greater the position reference degree, that is, the greater the reference degree of the second detection parameter.

In 550, in response to a condition that the first detection parameter is converted into a second electrical signal, and sent to the driving device and the current adjustment part, the first detection parameter may be used as a new second detection parameter, and the predicted reference point may be used as the predicted historical reference point corresponding to the new second detection parameter.

It should be understood that the operation numbers of operation 540 and operation 550 are merely intended for illustration, and do not limit the sequence of the operations. For example, operation 540 may be implemented after operation 550. In some embodiments, operation 540 and operation 550 may be unnecessary, that is, the process 500 may not include operation 540 and operation 550, or only include one of the operations.

In some embodiments, the controller 740 may determine a first sensitive coefficient of an object, determine a second sensitivity coefficient of a nerve, and determine a maximum elasticity value and a maximum current value of a current adjustment part based on the first sensitive coefficient and the second sensitive coefficient.

The first sensitive coefficient may be used to describe sensitivity of an object. The more sensitive the object is to stimulation, the greater the first sensitivity coefficient may be. In some embodiments, the first sensitive coefficient may be determined according to clinical manifestations of different objects. For example, corresponding clinical tests may be performed on the object before surgery, and the first sensitivity coefficient may be determined according to a test result of the object.

The second sensitive coefficient may be used to describe sensitivity of a nerve. The more sensitive the nerve is, the greater the second sensitivity coefficient may be. In some embodiments, the second sensitive coefficient may be determined according to a type of a nerve. For example, for a brain nerve, the second sensitive coefficient may be set to a larger value. For nerves in hands, feet, and knees, the second sensitive coefficient may be set to a smaller value.

In some embodiments, the controller 740 may determine a third sensitivity coefficient based on the first sensitive coefficient and the second sensitive coefficient. The third sensitivity coefficient may comprehensively reflect sensitivity of an object and a nerve. For example, the third sensitive coefficient may be determined based on the following equation (3).

c=k*a+m*b (3)

where a denotes a first sensitive coefficient; b denotes a second sensitive coefficient; c denotes a third sensitive coefficient; k and m denote weights of the first sensitive coefficient and the second sensitive coefficient, respectively. k and m may be determined based on experience, for example, both k and m may be 0.5.

In some embodiments, the controller 740 may determine a maximum elasticity value and a maximum current value of a current adjustment part based on the third sensitivity coefficient. For example, based on historical data, according to the third sensitivity coefficient, a maximum elasticity value, used by a patient with the same third sensitivity coefficient as the object in historical patients, and a maximum current value of the current adjustment part may be selected.

In some embodiments, the controller 740 may obtain relevant information of an object, and determine a first sensitivity coefficient of the object based on the relevant information of the object.

The relevant information may refer to information related to the object and helpful for judging the sensitivity degree of the object. For example, when the nerve is a nerve in a thyroid region of an object, the relevant information of the object may include occupational information and/or sound pressure level of speech.

The occupational information may refer to an occupation of the object, such as a soldier, a lawyer, a teacher, etc.

The sound pressure level of speech may refer to a sound pressure level of the sound of the object during daily conversation, usually expressed in decibels.

In some embodiments, the controller 740 may determine the first sensitive coefficient of the object based on the relevant information of the object. For example, when the occupation of the object is a teacher, a frequency of daily speech (teaching to students) of the object is higher, the sensitivity of the nerve in the laryngeal region of the object may be generally higher, and the first sensitivity coefficient of the object may be greater. As another example, when the sound pressure level of the object is high, the sensitivity of the nerve in the laryngeal region of the object may be generally higher, and the first sensitivity coefficient of the object may be greater.

In some embodiments of the present disclosure, prediction feedback and a prediction reference point corresponding to the first detection parameter may be output through a model based on the first detection parameter, the at least one group of the second detection parameters and the corresponding feedback signal, which can be provided to doctors for reference and helpful to locate a nerve faster and safer, thereby effectively preventing the nerve damage of patients due to improper operation during the operation. At the same time, the inherent relationship between a plurality of inputs in the model can be better built by using the Transformer model, which can improve prediction accuracy of the model.

It should be noted that the above description regarding the process 500 is merely provided for the purposes of illustration, and not intended to limit the scope of the present disclosure. For persons having ordinary skills in the art, multiple variations and modifications to the process 500 may be made under the teachings of the present disclosure. However, those variations and modifications do not depart from the scope of the present disclosure. For example, the process 500 may also include pre-processing operations.

FIG. 6 is a structural diagram illustrating an exemplary prediction model according to some embodiments of the present disclosure.

A prediction model 610 may refer to a machine learning model used to determine prediction feedback corresponding to a first detection parameter.

As shown in FIG. 6, input of the prediction model 610 may include a first detection parameter 601, and at least one group of second detection parameters and a corresponding feedback signal 602, and the output may include a feedback signal 621 corresponding to the first detection parameter.

In some embodiments, the output of the prediction model 610 may also include a prediction reference point 622.

In some embodiments, the input of the prediction model 610 may also include a position reference degree 603 corresponding to at least one group of second detection parameters.

In some embodiments, the prediction model 610 may be obtained through training. For example, training samples may be input into an initial prediction model 611, and a loss function may be constructed based on the output of the initial prediction model 611. The parameters of the initial prediction model 611 may be iteratively updated based on the loss function until the preset conditions are satisfied and the training is completed.

In some embodiments, training samples 620 may include at least one group of historical second detection parameters and the corresponding feedback signal, the historical first detection parameter, and the position reference degree corresponding to the at least one group of historical second detection parameters. Labels 630 may be a feedback signal corresponding to the historical first detection parameter. Training samples and labels may be obtained based on historical data.

In some embodiments of the present disclosure, the second detection parameter and the corresponding feedback signal, and the first detection parameter may be processed based on the prediction model. More and richer intrinsic features may be extracted from the information of a plurality of historical detections for prediction feedback corresponding to the first detection parameter and prediction of prediction reference points. The prediction model may be obtained by iteratively updating the parameters of the initial prediction model based on the loss function, and the training may be completed when the preset conditions are satisfied, so that the model may have stronger adaptive and predictive capabilities.

FIG. 7 is a structural diagram illustrating an exemplary neuromonitoring device according to some embodiments of the present disclosure.

In some embodiments, the neuromonitoring device 700 may include a probe 710, an operation part 720, a display part 730, a controller 740, a probe monitoring part 750, a fixing device 760, and a driving device 770.

The probe 710 may include a probe head, an elastic piece and an elastic measuring piece. The probe head may be connected with the elastic piece. The elastic measuring piece may be connected to the elastic piece and may be used to measure an elasticity value of the elastic piece and convert the elasticity value into a first electrical signal.

In some embodiments, the probe 710 may also include a sleeve. The elastic piece may be installed in the sleeve. An end of the probe head is inserted into a first end of the sleeve to connect to the elastic piece. A second end of the sleeve may be connected to the handle.

In some embodiments, the probe 710 may also include an electromyography measuring piece used to measure a second electrical signal. The second electrical signal may be an electromyographic signal of an object of the probe.

In some embodiments, the operation part 720 may include a handle. The probe 710 may be fixedly connected to the handle, and the display part 730 may be set on the handle.

In some embodiments, the operation part 720 may be used to receive a control operation.

The display part 730 may be used to display prompt information. The prompt information may include prompt information regarding the elasticity value determined based on the first electrical signal. The prompt information may also include a magnitude of a nerve stimulation current.

The controller 740 may be used to receive a control operation from the operation part to determine action instructions and send the action instructions to the driving device to drive the probe to move.

In some embodiments, the controller 740 may be used to adjust action instructions sent to the driving device when a movement range or speed of the control operation does not meet a preset condition.

In some embodiments, the controller 740 may be used to measure a second electrical signal. The second electrical signal may be an electromyographic signal of an object of the probe.

In some embodiments, the controller 740 may be used to display prompt information. The prompt information may include electromyographic information based on the second electrical signal.

In some embodiments, the controller 740 may be used to determine a first sensitive coefficient of the object, determine a second sensitivity coefficient of a nerve, and determine a maximum elasticity value and a maximum current value of a current adjustment part based on the first sensitive coefficient and the second sensitive coefficient.

In some embodiments, the controller 740 may be used to obtain relevant information of the object. The relevant information may include occupational information and/or a sound pressure level of speech. The first sensitive coefficient of the object may be determined based on the relevant information of the object.

In some embodiments, the controller 740 may be used to obtain a first detection parameter, and at least one group of second detection parameters and a corresponding feedback signals; and process the first detection parameter, the at least one group of second detection parameters and the corresponding feedback signal based on a prediction model to output prediction feedback corresponding to the first detection parameter. The prediction model may be a machine learning model.

In some embodiments, the controller 740 may be used to process the first detection parameter, the at least one group of second detection parameters and the corresponding feedback signal based on the prediction model to output a prediction reference point. The prediction reference point may be a point in the object related to the feedback signal; and the prediction reference point may be used to display.

In some embodiments, the second detection parameter may include an operating pressure, an operating current, and an operating position. The controller 740 may determine, based on a distance between the operating position and a predicted historical reference point corresponding to the second detection parameter, a position reference degree of the second detection parameter, and process the first detection parameter, the at least one group of second detection parameter and the corresponding feedback signal, the at least one group of second detection parameter and the corresponding position reference degree based on the prediction model to output the prediction reference point.

In some embodiments, the controller 740 may determine warning information according to the prediction feedback, and display the warning information.

The probe monitoring part 750 may be used to monitor a usage status of the probe and generate probe monitoring information. The usage status of the probe 710 may include a cumulative usage time of the probe 710 and/or an elastic condition of the elastic piece.

The fixing device 760 may be used to fix positional relationship between the driving device 770 and an object. The driving device 770 may be used to drive the probe to move based on the control operation.

Some embodiments of the present disclosure may also disclose a computer-readable storage medium storing computer instructions. When reading the computer instructions in the storage medium, a computer implements the following method. The method may include receiving a control operation from an operation part, and determining action instructions and sending the action instructions to a driving device to drive a probe to move.

The advantage effects of the embodiments of the present disclosure may include but not limited to: (1) an elastic piece is set to make a probe head retractable, which can ensure a reliable contact between the probe head and nerves or tissues; (2) the elastic piece also allows a user to sense a resilience force, in combination with an elastic prompt which can prompt an elasticity value, the user can know a pressure applied to a patient by the probe head during operation so as to adjust a strength in time to further ensure the reliable contact between the probe head and the nerves or the tissues and protect the nerves or the tissues from injury; (3) for different types of nerves or tissues, or for individuals with different sensitivities, neuromonitoring devices with different maximum elasticity values may be used, or appropriate maximum elasticity values may be adjusted, which can ensure the nerves or the tissues are not damaged by excessive pressures exerted by the probe under a premise of ensuring a detection effect; (4) for different types of nerves or tissues, or for individuals with different sensitivities, the magnitude of the stimulation current may be adjusted to achieve a better detection effect. It should be noted that different embodiments may have different advantage effects. In different embodiments, the advantage effects may include any combination of one or more of the above or any other possible advantage effect.

The basic concept has been described above, and it is obvious to those skilled in the art that the detailed disclosure is merely exemplary and does not constitute a limitation of the present disclosure. Various alterations, improvements, and modifications to the present disclosure may be made by those skilled in the art, although not explicitly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, certain features, structures, or features of one or more embodiments of the present disclosure may be combined as appropriate.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure method does not mean that the present disclosure object requires more features than the features mentioned in the claims. Rather, claim subject matter lie in less than all features of a single foregoing disclosed embodiment.

We claim:

1. A neuromonitoring device, comprising a probe, an operation part, and a display part; wherein
the probe includes a probe head, an elastic piece, and an elastic measuring piece; the probe head is connected to the elastic piece; the elastic measuring piece is connected to the elastic piece and is configured to measure an elasticity value of the elastic piece and convert the elasticity value into a first electrical signal; and
the display part is configured to display prompt information, wherein the prompt information includes prompt information regarding the elasticity value determined based on the first electrical signal.

2. The neuromonitoring device of claim 1, wherein the operation part includes a handle, the probe is fixedly connected to the handle, and the display part is set on the handle.

3. The neuromonitoring device of claim 1, wherein the prompt information includes a magnitude of a nerve stimulation current.

4. The neuromonitoring device of claim 1, further comprising a probe monitoring part configured to monitor a usage status of the probe and generate probe monitoring information, wherein the usage status of the probe includes a cumulative usage time of the probe and/or an elastic condition of the elastic piece.

5. The neuromonitoring device of claim 2, wherein
the probe further includes a sleeve,
the elastic piece is installed in the sleeve,
an end of the probe head is inserted into a first end of the sleeve to connect to the elastic piece and a second end of the sleeve is connected to the handle.

6. The neuromonitoring device of claim 1, further comprising a fixing device and a driving device, wherein
the operation part is configured to receive a control operation,
the driving device is configured to drive the probe to move based on the control operation, and
the fixing device is configured to fix positional relationship between the driving device and an object.

7. The neuromonitoring device of claim 6, further comprising a controller configured to adjust action instructions sent to the driving device when a movement range or speed of the control operation does not meet a preset condition.

8. The neuromonitoring device of claim 6, further comprising an electromyography measuring piece configured to measure a second electrical signal, wherein the second electrical signal is an electromyographic signal of the object of the probe.

9. The neuromonitoring device of claim 8, wherein the prompt information includes electromyographic information based on the second electrical signal.

10. A neuromonitoring method implemented by a nerve detection device, comprising:
receiving a control operation from an operation part; and
determining action instructions and sending the action instructions to a driving device to drive a probe to move.

11. The method of claim 10, further comprising adjusting the action instructions sent to the driving device when a movement range or speed of the control operation does not meet a preset condition.

12. The method of claim 11, further comprising measuring a second electrical signal, wherein the second electrical signal is an electromyographic signal of an object of the probe.

13. The method of claim 12, further comprising displaying prompt information, wherein the prompt information includes electromyographic information based on the second electrical signal.

14. The method of claim 13, further comprising
determining a first sensitivity coefficient of the object;
determining a second sensitivity coefficient of a nerve; and
determining a maximum elasticity value and a maximum current value of a current adjustment part based on the first sensitivity coefficient and the second sensitivity coefficient.

15. The method of claim 14, wherein the determining a first sensitivity coefficient of the object includes:
obtaining relevant information of the object, the relevant information including occupational information and/or a sound pressure level of speech; and
determining, based on the relevant information of the object, the first sensitivity coefficient of the object.

16. The method of claim 11, further comprising
obtaining a first detection parameter, at least one group of second detection parameters and a feedback signal corresponding to the at least one group of the second detection parameters; and
processing the first detection parameter, the at least one group of second detection parameters and a feedback signal corresponding to the at least one group of the second detection parameters based on a prediction model to output prediction feedback corresponding to the first detection parameter, wherein the prediction model is a machine learning model.

17. The method of claim 16, further comprising processing the first detection parameter, the at least one group of second detection parameters and a feedback signal corresponding to the at least one group of the second detection parameters based on the prediction model to output a prediction reference point, wherein the prediction reference point is a point in the object related to the feedback signal; and the prediction reference point is configured to display.

18. The method of claim 17, wherein for one of the at least one group of second detection parameters, the second detection parameter includes: an operating pressure, an operating current, and an operating position, and the method further includes:
determining, based on a distance between the operating position and a predicted historical reference point corresponding to the second detection parameter, a position reference degree of the second detection parameter; and
processing the first detection parameter, the at least one group of second detection parameters and a feedback signal corresponding to the at least one group of the second detection parameters, the at least one group of second detection parameters and the corresponding position reference degree based on the prediction model to output the prediction reference point.

19. The method of claim 16, further comprising
determining warning information according to the prediction feedback; and
displaying the warning information.

20. A computer-readable storage medium storing computer instructions, wherein when reading the computer instructions in the storage medium, a computer implements the method including:
receiving a control operation from an operation part; and
determining action instructions and sending the action instructions to a driving device to drive a probe to move.
